(19) United States
(12) Patent Application Publication
(10) Pub. No.: US 2022/0276245 A1
(43) Pub. Date: Sep. 1, 2022

(54) TEST SYSTEM FOR RECOGNIZING LEGIONELLAE

(71) Applicants: SECURETEC DETEKTIONS-SYSTEME AG, Neubiberg (DE); TECHNISCHE UNIVERSITÄT MÜNCHEN, Munich (DE)

(72) Inventors: Torsten STADTHAGEN, Munich (DE); Patrick GRUBER, Munich (DE); Hannelore MEYER, Munich (DE); Nina WANTIA, Munich (DE)

(21) Appl. No.: 17/633,791

(22) PCT Filed: Aug. 10, 2020
(86) PCT No.: PCT/EP2020/072438

§ 371 Date: Feb. 8, 2022

(30) Foreign Application Priority Data
Aug. 9, 2019 (DE) 10 2019 121 588.9

(51) Int. Cl. G01N 33/569; G01N 33/558

(52) U.S. Cl. CPC G01N 33/56911; G01N 33/558; G01N 2469/10

(57) ABSTRACT

The present disclosure relates to a test system for identifying legionellae. Furthermore, the present disclosure relates to a test system for detecting an infection with legionellae, and to the use of a test system in a method for detecting legionellae. The disclosure further relates to a method for detecting an infection with legionellae.

### TECHNICAL FIELD

The present disclosure relates to a test system for recognizing legionellae. The present disclosure also relates to a test system for detecting an infection with legionellae as well as to the use of a test system in a method for detecting legionellae. The disclosure also relates to a method for detecting an infection with legionellae.

### BACKGROUND AND SUMMARY

At present, specific pathogen diagnostics must be performed to detect legionellosis since the clinical picture is non-specific and thus does not allow a conclusion to be drawn about the pathogen. According to the prior art, legionellosis can be detected by a number of methods.

For the time being, legionellae are detected mainly (in 73% of cases) by the immunological detection of lipopolysaccharide (LPS). However, the sensitivity of these tests, which is 75%, is not satisfactory which is why a legionellae infection cannot be reliably excluded if the result is negative. Furthermore, the tests available on the market can only detect an infection with Legionella pneumophila of serogroup 1. Although Legionella pneumophila is the causative agent of severe legionella diseases in more than 90% of cases, the serogroup is causative in only two-thirds of cases. In particular, nosocomial infections often involve strains of other pathogen groups that cannot be detected by this method.

Alternatively, PCR-based tests from respiratory samples (usually bronchial lavage) are carried out. Although sensitivity and specificity are high, these diagnostic methods are, however, complicated, expensive, and limited in use due to the invasive sampling which involves a significant risk of subsequent patient intubation. In addition, highly qualified and trained personnel are required to detect legionellae via PCR, as a result of which these methods are mainly considered for clinical use.

Serological tests detect antibodies against legionellae in the patient's serum, which, however, only become detectable after 3 to 10 weeks following an infection. Due to its low sensitivity, this method is not suitable for rapid diagnostics.

Other methods for a direct detection of pathogens (microscopy, culture) only play a minor role and are of limited use due to the complicated sampling, performance or long culture duration (several days).

According to an overview on legionellosis by the Robert-Koch-Institute from the end of March 2015, the antigen test from urine is the most frequently used test (73.1%), followed by the PCR method (12.8%), serological detection (9.3%) and direct pathogen detection in culture with 4.4% (based on 841 mentions in 806 cases).

All in all, it can be stated that no suitable test is available for the reliable and rapid detection of a legionella infection for clinical use or diagnosis by a family doctor.

Legionnaires' disease (legionellosis) is a severe and, if left untreated, often fatal bacterial pneumonia but it represents only a small portion of atypical pneumonias. For the detection of the bacterial pathogens, the legionellae, a diagnostic procedure on the basis of a lateral flow test (LFT) shall be developed, which surpasses the previous diagnostic procedures in terms of sensitivity and specificity as well as test speed. The administration of macrolide and fluoroquinolone antibiotics, which has so far been instigated in the case of pneumonia and is unnecessary due to the rarity of legionellosis, shall thus be avoided. Saliva shall be used as sample material because of its easy, non-invasive availability. This can, among other things, reduce the development of resistance as well as support the diagnosis already at the family doctor.

Legionellosis is a severe form of pneumonia caused by the bacterium Legionella pneumophila. Every year, about 200,000 patients with pneumonia are treated in hospital in Germany. These patients are examined as a standard with regard to a legionella infection. Until a result is available, broad-spectrum antibiotics are administered as a preventive measure. The aim of the project is to develop a reliable rapid test for the detection of legionellae in saliva. The present disclosure shall provide a rapid test result with easy handling and non-invasive sampling. The field of application shall also extend to countries with weak medical infrastructure.

The object of the present disclosure is to provide a test system for the detection of an infection with legionellae, in particular a test system which allows reliable and rapid detection.

According to the disclosure, this object is achieved by a test system. According thereto, a test system shall be provided to identify legionnaires' disease.

Legionnaires' disease can alternatively be referred to as legionellosis.

The disclosure is based on the fundamental idea that a prophylactic administration of antibiotics against legionellosis, or the administration of antibiotics due to false negative test results from unreliable tests, shall be reduced and/or avoided, and thus the development of antibiotic resistance can be at least partially counteracted.

The test system can alternatively and/or additionally be set up for detecting an infection of a subject with legionellae. A subject can here be a mammal, particularly a human. In particular, the subject can be a patient. In particular, it is possible for the test system to also detect an infection of a subject before the disease (legionellosis) breaks out. Again, a significant advantage is that a prophylactic administration of antibiotics against legionellosis shall be reduced and/or avoided and thus the development of antibiotic resistance can be at least partially counteracted.

In one embodiment, the test system is or comprises a lateral flow test (LFT). In particular, a lateral flow test is understood to be an immunological method for the qualitative detection of substances with corresponding binding partners. A lateral flow test of this type can also be made at any conceivable location, in particular no laboratory environment and no laboratory personnel or medically trained personnel are required. A lateral flow test thus allows a quick, simple and inexpensive test to be carried out to detect an infection with legionellae or to identify legionellosis. This also makes it possible to support the family doctor in the diagnosis. The test duration from sampling can be less than one hour, in particular less than 30 minutes.

The test system can be set up to identify a legionnaires' disease and/or to detect an infection with legionellae in saliva and/or sputum. Therefore, the test system can be used in an user-friendly way or the method can be carried out in a user-friendly way without an invasive collection of a sample (e.g. respiratory tract samples obtained via bronchial lavage, blood or lymph) from the subject to be tested. The test system according to the disclosure may also be used over test systems that are set up for the detection of pathogens in urine and/or stool because toilets and associated intimate activities are not required for the use of the test system according to the disclosure. It is generally conceivable that the saliva sample or sputum sample comprises a freshly collected sample or a stored, cooled or frozen sample.

However, the use of blood, respiratory tract samples (especially of the lower respiratory tract), lymph, urine and/or other body fluids and/or stool is alternatively and/or additionally conceivable and/or possible. In particular, the sample can be used as an untreated sample or a pre-treated sample.

In particular, it is possible that the identification and/or detection is conducted at least in part via the binding of at least one protein, in particular a protein of the bacterial genus Legionella, to a specific binding partner. In particular, the protein can also be referred to as a marker protein. A high specificity of the test system is thus achieved.

In one embodiment, the test system is a test system for detecting legionellosis in a saliva sample, the test system comprising a lateral flow test and the detection of the marker FLA by the test system being indicative of the presence of legionellosis.

In one embodiment, the test system is a test system for detecting legionellosis in a saliva sample, the test system comprising a lateral flow test and the detection of the marker MIP by the test system being indicative of the presence of legionellosis.

In one embodiment, the test system is a test system for detecting legionellosis in a saliva sample, the test system comprising a lateral flow test and the detection of the marker PAL by the test system being indicative of the presence of legionellosis.

It is also possible to combine two or more markers in one test system.

In this regard, a protein can comprise a peptide, lipoprotein, glycoprotein, peptidoglycan, filament, antigen and/or epitope.

Generally, it is possible that the at least one protein is a protein of the species Legionella pneumophila, Legionella micadei, Legionella bozmanii, Legionella dumoffii and/or Legionella longbeachae. In particular, it is also possible to identify and/or detect different and/or all (of the about 57) serotypes of Legionella pneumophila with the test system, especially serotypes 1, 4 and/or 6. This has the advantage that not only a disease/infection attributable to Legionella pneumophila serotype 1—the most common causative agent of legionnaires' disease—can be detected and/or identified. Thus, false negative results are avoided by tests specific for only one serotype.

It is conceivable that the at least one protein or also several proteins comprise macrophage inflammatory protein (MIP and/or (Legionella pneumophila) peptidoglycan-associated lipoprotein (PAL) and/or flagellin (FLA).

Macrophage inflammatory protein (MIP) is a 24-kDa protein (pI 9.8). MIP can comprise MIP-1 alpha and/or MIP-1 beta.

(Legionella pneumophila) peptidoglycan-associated lipoprotein (PAL) is one of the most highly conserved proteins of legionellae, achieving sensitivity and specificity values of about 90% in diagnostic applications.

Flagellin (FLA) is a component of the flagellum of Legionella pneumophila and is also involved in host cell invasion.

It is also generally conceivable to use other marker proteins of high conservation, prevalence, and immunogenicity for the detection of a legionella infection and/or the identification of legionnaires' disease. In particular, surface proteins are conceivable.

In one embodiment, the test system comprises a test element comprising microfluidic structures, such as a chromatographic test strip. The test element optionally comprises a first region set up for absorbing eluent, a second region set up for applying a sample, a third region set up for detecting, such as optically detecting, the protein, in particular MIP, PAL and/or FLA, optionally a fourth region set up for absorbing excess eluent. The test system can further optionally comprise a housing. A test system of this type has the advantage of being very compact, thus greatly facilitating the application of the test. The housing additionally protects against external interference factors, such as damage to the test or contamination. In addition, only a very small sample volume of 1-20 μl saliva, in particular 2-16 μl saliva, in particular 4 μl saliva, is required.

In a further embodiment, the test element can comprise a further (fifth) region, the further (fifth) region comprising, at least temporarily, the specific binding partner for the protein, in particular MIP, PAL and/or FLA.

The test system and/or test element can comprise a porous material, in particular a fibrous web of cellulose, glass fiber, polymer fiber, nylon, silk, wool, felt and/or cotton linter and/or a sponge.

In a further embodiment, the test system also comprises a control system, particularly an optical control system. In particular, the control system can also be referred to as a control unit or control element. The control system can indicate whether the test system has been used in such a way that an identification of legionellosis and/or detection of legionellae is technically possible. In other words, the likelihood of false results due to incorrectly performed tests using the test system is reduced. In particular, the control system can comprise the specific binding partner of the at least one protein that is associated with the labeling element and/or at least one specific binding partner of the specific binding partner of the at least one protein. In particular, the control system can comprise at least one antibody.

The test system can further comprise a sampling element set up for receiving a sample containing the protein. For example, a sampling element can be a saliva collector and/or sputum collector. Thus, the identification and/or detection of legionellosis and/or an infection with legionellae can be performed from sampling to identification/detection with only one test system, which is an safe, fast and simple application of the test system.

The disclosure further relates to the use of a test system for detecting an infection with legionellae.

Furthermore, the disclosure relates to a method for detecting legionellae in a sample, comprising the following steps:

(a) providing a test system,

(b) applying the sample to a test element, and

(c) detecting the presence of at least one protein, in particular MIP, PAL and/or FLA in the sample,

(d) the test system comprising at least one specific binding partner to which the at least one protein, in particular MIP, PAL and/or FLA, binds, the detection being effected at least in part via the binding of the at least one protein, in particular MIP, PAL and/or FLA, to the specific binding partner.

In one embodiment, the method according to the disclosure is a method for detecting legionellosis in a saliva sample, a lateral flow test being carried out, and the proof and/or the detection of the marker FLA being indicative of the presence of legionellosis.

In another embodiment, the method according to the disclosure is a method for detecting legionellosis in a saliva sample, a lateral flow test being performed and the proof and/or the detection of the marker MIP being indicative of the presence of legionellosis.

In another embodiment, the method according to the disclosure is a method for detecting legionellosis in a saliva sample, a lateral flow test being performed and the proof and/or the detection of the marker PAL being indicative of the presence of legionellosis.

It is generally possible that the method further comprises lysing the bacteria in the sample. This has the advantage that the method and/or the test system, where applicable, becomes more susceptible and/or more sensitive.

Some bacterial proteins, for example MIP and PAL, are localized in/on the cell membrane of the bacteria (legionellae) and partially covered by the lipopolysaccharide. Therefore, contact with specific binding partners on the intact bacterium is sterically at least partially hampered. Opening the cell membrane (lysing) can make the targets, i.e. the proteins (legionella proteins) to which the specific binding partners shall bind, more accessible.

In general, it is possible for the sample to comprise a saliva sample or sputum. In other words, the test system can be set up to identify and/or detect legionellae in saliva and/or sputum. The advantages of a test system of this type have already been mentioned.

It is conceivable, for example in a further method step, that in order to prevent degradation of the marker proteins in saliva, enzyme inhibitors are added to the saliva samples (analogously Fung et al. “Quantitative detection of Pf HRP2 in saliva of malaria patients in the Philippines”, Malaria Journal, 2012, 11:175). For example, the use of an inhibitor cocktail of aprotinin, PMSF, and sodium orthovanadate is here conceivable. Other inhibitors are also conceivable in general, especially protease inhibitors.

The specific binding partner can comprise a monoclonal antibody, polyclonal antibody, affinity-purified antibody, antibody fragment, peptide, protein, nanobody, nucleic acid fragment, aptamer, anticalin, lectin, affibody and/or chemical ligand, the specific binding partner being linked to a labeling element. In other words, a conjugate of specific binding partner and labeling element is present. The association of a specific binding partner for at least one protein (marker protein) with a labeling element renders possible the detection and/or identification, in particular optical detection and/or optical identification.

The labeling element can comprise a dye, a fluorochrome, fluorophore, luminophore, enzyme, peptide, biotin, oligonucleotide, protein tag, radioactive isotope, non-radioactive nuclide, nanoparticle, in particular gold nanoparticle, fluorescent nanoparticle, magnetic nanoparticle and/or quantum dot. Thus, the ability of a binding partner specific to a marker protein to bind to a corresponding marker protein and the ability of a labeling element to provide a detectable and/or identifiable signal can be present in combination. In particular, an association of a specific binding partner of a marker protein with a nanoparticle, e.g. a gold nanoparticle, can have the advantage of being detectable to the naked eye.

A “nanoparticle” or “nanocorpuscle” can refer to a composite of a few to a few thousand atoms or molecules, the size of which is typically from 1 to 200 nanometers, but can be extended to 1000 nanometers, if necessary. However, the nanoparticle can also have a size <1 nm.

The nanoparticle can be a plastics, natural material or metallic nanoparticle, in particular a gold nanoparticle (also referred to as nanogold). In general, it is also possible that gold nanoparticles are colloidal gold nanoparticles.

In a particularly embodiment, the specific binding partner is an antibody and the labeling element is a nanoparticle, in particular gold nanoparticle (also referred to as nanogold).

In particular, it is conceivable that the specific binding partner is available in soluble form.

In particular, the specific binding partner can be temporarily immobilized on the test element, in particular in a defined region of the test element.

In a further embodiment, the specific binding partner is present in dried form.

To date, two different legionella marker proteins have been produced in recombinant manner Various antibodies have been generated against these marker proteins and tested for their applicability in lateral flow technology. Initial experimental setups show that the simple and sensitive detection of both markers is possible in the lateral flow format. Further steps will be the optimization of the test with regard to sensitivity and a feasibility study with patient samples.

Furthermore, another legionella marker protein was produced recombinantly.

Advantages:

PoC saliva test (point-of-care saliva test) can reduce the prophylactic administration of antibiotics against legionellosis and thus counteract the development of resistance.

Support of the family doctor in the diagnosis.

Simple, user-friendly handling and non-invasive sampling. This makes the test system suitable for use even in regions with a less developed medical infrastructure.

Rapid diagnosis since the measurement result is available after a maximum of 30 minutes.

### BRIEF DESCRIPTION OF THE FIGURES

Further details of the disclosure will now be explained with reference to an exemplary embodiment shown in more detail in the drawings.

In these drawings:

FIG. 1 shows a schematic drawing illustrating the use of the test system according to the disclosure;

FIG. 2 shows a further schematic drawing illustrating the use of the test system according to the disclosure;

FIG. 3 schematically shows the basic operating principle of the test system;

FIG. 4 shows the use of the marker proteins;

FIG. 5 shows the characterization of poly- and monoclonal antibodies;

FIG. 6 shows the structure of a lateral flow test strip;

FIG. 7 schematically shows the operating principle of an LFT system used in the test system according to the disclosure;

FIG. 8 shows the production of conjugation of gold and antibodies;

FIG. 9 shows the stability of antigens in saliva;

FIG. 10 shows the specificity of polyclonal α-MIP and α-PAL;

FIG. 11 shows an exemplary laboratory pattern of a lateral flow test according to the disclosure;

FIG. 12 shows exemplary test systems for MIP each comprising a control system;

FIG. 13a shows by way of example the selection of antibody pairings for PAL for use in a test system according to the disclosure;

FIG. 13b shows by way of example the selection of antibody pairings for PAL for use in a test system according to the disclosure on the basis of a test membrane;

FIG. 14 shows by way of example the selection of antibody pairings for MIP for use in a test system according to the disclosure;

FIG. 15 shows exemplary conditions for the conjugation of polyclonal antibodies and nanogold; and

FIG. 16 schematically shows a method according to the disclosure for the detection of legionellae in a sample.

### DETAILED DESCRIPTION

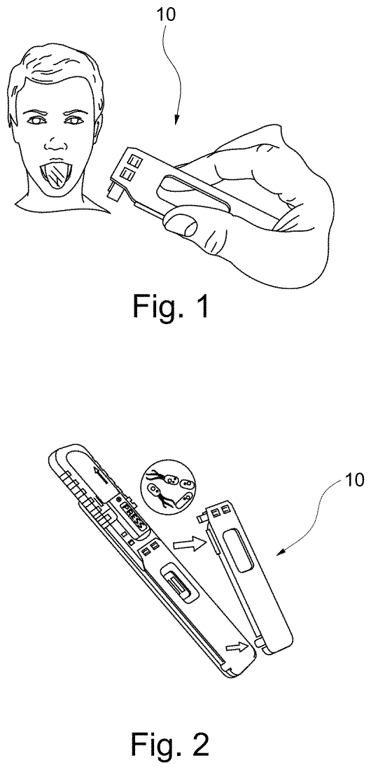

FIG. 1 shows a schematic drawing illustrating the use of the test system 10 according to the disclosure. Here, saliva from a possible patient is taken in the region of the oral cavity or in another suitable manner

In particular, the sampling is shown by a sampling element.

In general, the saliva can be taken by a sampling element, for example a wiper, or the test system itself.

In this case, the test system 10 is a lateral flow test (LFT).

Alternatively, a sputum sample can be used, for example.

FIG. 2 shows a further schematic drawing illustrating the use of the test system 10 according to the disclosure. The saliva droplet is applied to a saliva collector, for example, and is applied with this collector to the lateral flow test LFT.

In other words, the test system 10 here comprises a lateral flow test LFT.

The saliva collector can also be referred to as a sampling element.

In other words, the test system 10 here comprises a sampling element.

Shown here is a test system 10 comprising two test strips 12.

In general, a test system 10 can also comprise only one test strip 12 or more than two test strips 12.

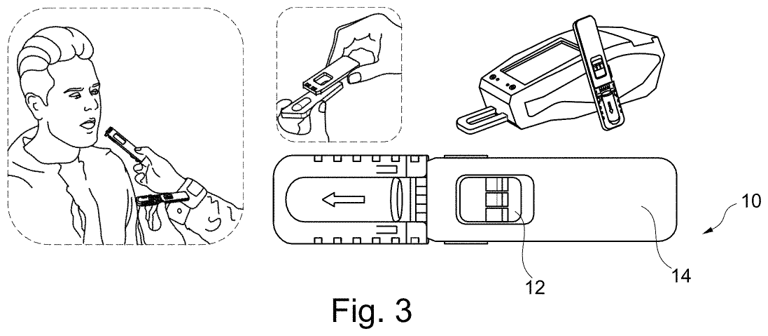

FIG. 3 schematically shows the basic operating principle of the test system 10, which is designed here as a lateral flow test (LFT).

The proven DrugWipe® technology from Securetec Detektions-Systeme AG is used for the highly sensitive and rapid PoC detection. The DrugWipe® rapid saliva test has been used very successfully by police and customs worldwide for the detection of drugs in saliva for more than 20 years. This technology is adapted to the detection of legionellae in saliva.

In other words, the test system 10 is a test system 10 for the identification of legionnaires' disease.

The test system 10 is set up to identify legionnaires' disease and/or to detect an infection with legionellae in saliva and/or sputum.

The detection can be carried out at least in part via the binding of at least one protein M, in particular a protein of the bacterial genus Legionella, to a specific binding partner B.

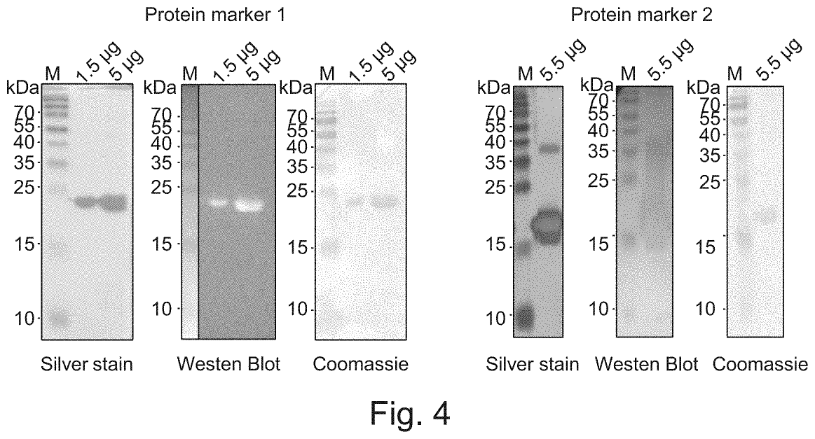

The protein M can also be referred to as a marker protein, cf. FIG. 4.

FIG. 4 shows the use of the marker proteins.

Three marker proteins (M1-M3) were selected on the basis of distribution, immunogenicity and homology between Legionella pneumophila strains. M1 and M2 were generated as recombinant proteins in E. coli and purified at mg scale. They are used to obtain antibodies.

M1 can be MIP (macrophage inflammatory protein), in particular MIP 1-alpha (macrophage inflammatory protein 1-alpha).

M2 can be PAL.

M3 can be FLA.

The polyclonal antibodies α-MIP and α-PAL have shown high sensitivity and specificity.

They form stable conjugates (immunoconjugates) with gold particles N.

MIP and PAL are also stable in saliva.

The same is true for FLA.

For experimental purposes in testing the disclosure, MIP (23 mg) and PAL (61 mg) were produced with >95% purity and detected with the corresponding antibodies (i.e. MIP and PAL).

Results to date:

marker proteins successfully produced in recombinant manner (MIP, PAL, FLA);

antibodies developed;

detection of 10 ng/ml marker protein on lateral flow test strips (prototypes).

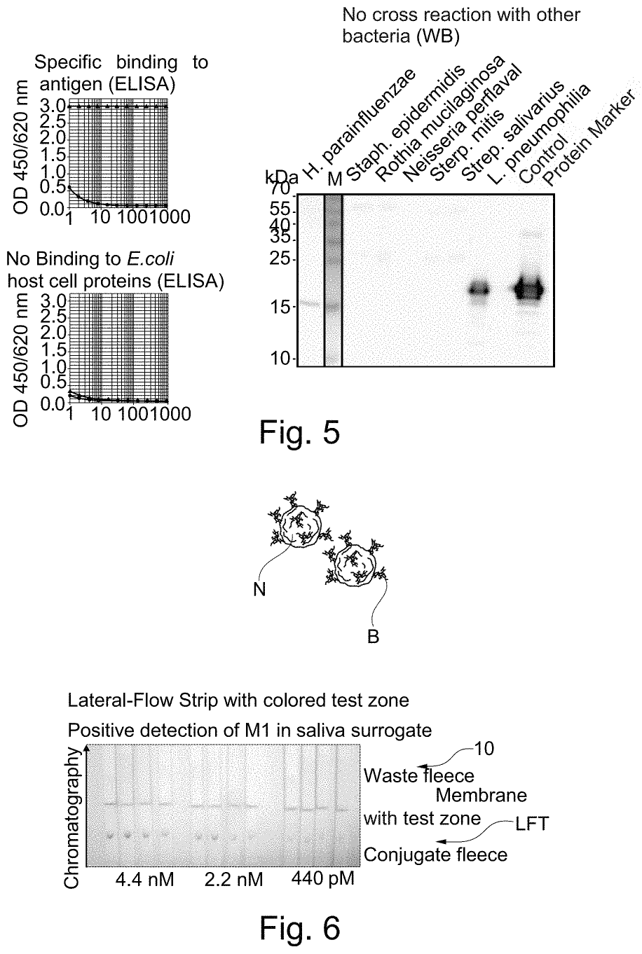

FIG. 5 shows the characterization of poly- and monoclonal antibodies.

To date, 7 antibodies each against M1 (MIP) and M2 (PAL) have been generated and selected for specificity, sensitivity and stability.

The exclusion of cross-reactions ensures that LegioWipe does not identify other pathogens of pneumonia or microorganisms of the normal oral flora.

The seven antibodies include polyclonal and monoclonal antibodies in each case.

Furthermore, antibodies against FLA were generated and selected for specificity, sensitivity and stability.

FIG. 6 shows the setup of a lateral flow test strip 12.

M1 (MIP) was detected in saliva surrogate.

Conjugation of the antibodies with gold nanoparticles results in red colored “nanoprobes” which allow the immunological detection of legionellae.

Initial laboratory samples achieved a sensitivity of approximately 10 ng/ml (440 pM) of marker protein.

Further laboratory samples achieved a sensitivity of approximately 2 ng/ml-20 ng/ml marker protein.

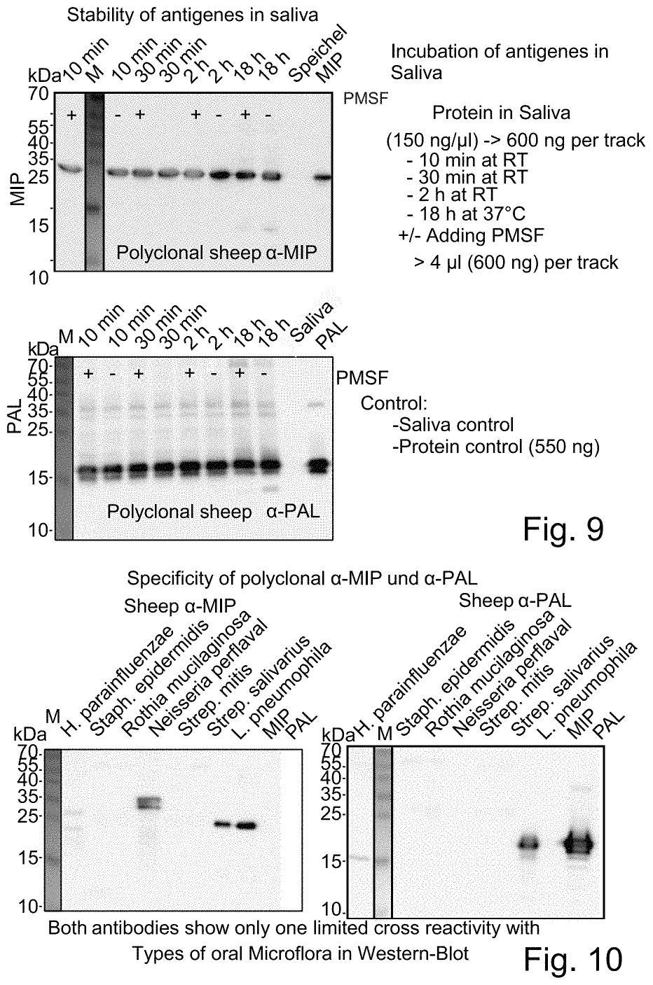

Model experiments also showed that the marker proteins are stable in human saliva, cf. FIG. 9.

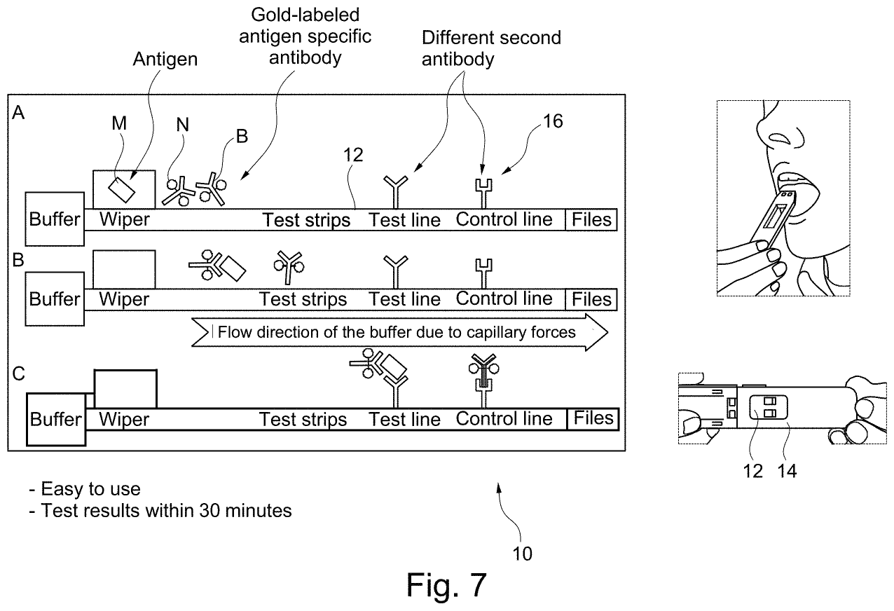

FIG. 7 schematically shows the operating principle of an LFT system used in the test system 10 according to the disclosure.

This is a lateral flow test LFT in sandwich format.

A sample to be tested (saliva in this example) is applied to the test element 12 (indicated here by the wiper, which can also be referred to as the sampling element).

After the addition of the solvent (here buffer), the sample starts to spread over the test element, 12 due to capillary forces (thin layer chromatography).

The sample migrates with the liquid to an area where immunoconjugates (antibody B with nanogold N) are located.

In this embodiment, the immunoconjugates are initially available in dried form.

The liquid detaches the immunoconjugates from the test element 12.

Specific binding of one immunoconjugate at a time to one protein to be detected in the sample, if present, is rendered possible.

The liquid continues to migrate into the capillary region where an antibody has been immobilized in a small section (may also be referred to as test line antibody) that binds to a different location on the surface of the marker protein to be detected than the immunoconjugate does, thereby binding and enriching the marker protein to be detected in that region while the liquid continues to migrate.

The enrichment of the protein M to be detected and the immunoconjugate bound thereto results in staining on the test element 12 on the basis of the bound immunoconjugate with the nanogold N, in this case in the form of a line.

Accordingly, the protein M is identified and/or detected at least in part via the binding of at least one protein M to a specific binding partner B.

The liquid migrates further into the capillary region, where in another small section another antibody has been immobilized (may also be referred to as control line antibody), which binds the immunoconjugate (directly) and thereby accumulates it in this region, while the liquid continues to migrate.

The accumulation of immunoconjugates results in staining on the test element 12 via the nanogold N, in this case in the form of a line.

This can also be referred to as a control system 16.

Alternatively, a lateral flow test LFT in competitive format is conceivable.

Detection by means of a non-competitive LFT with a negative indication is also conceivable.

Accordingly, the test system 10 comprises a test element 12 with microfluidic structures, in particular a chromatographic test strip 12.

In this exemplary embodiment, the test strip 12 comprises a first region set up to absorb eluent (in this example, a buffer).

In another exemplary embodiment, this first region can be absent.

In this exemplary embodiment, the test strip 12 comprises a second region set up for application of a sample.

In this exemplary embodiment, the test strip 12 comprises a third region set up for detecting, such as optically detecting, the protein M.

In this exemplary embodiment, the test strip 12 comprises a fourth region set up for the absorption of excess eluent (fleece).

In another exemplary embodiment, this fourth region can be absent.

The test strip can comprise a further (fifth) region, the further (fifth) region comprising, at least temporarily, the specific binding partner for the protein M, in particular MIP and/or PAL and/or FLA.

The test system 10 further comprises a housing 14.

In particular, the test element 12 is incorporated into a housing 14.

In another embodiment, the housing 14 can be absent.

FIG. 7 does not show that a specific binding partner B can generally comprise a monoclonal antibody, polyclonal antibody, affinity-purified antibody, antibody fragment, peptide, protein, nanobody, nucleic acid fragment, aptamer, anticalin, lectin, affibody, and/or chemical ligand.

FIG. 7 also fails to show that the labeling element N can generally comprise at least one dye, fluorochrome, fluorophore, luminophore, enzyme, peptide, biotin, oligonucleotide, protein tag, radioactive isotope, non-radioactive nuclide, nanoparticle, in particular gold nanoparticle, fluorescent nanoparticle, magnetic nanoparticle, and/or quantum dot.

Furthermore, FIG. 7 does not show that the specific binding partner B (for the protein M to be detected) is generally available in soluble form and/or dried (in dried form).

The illustrated lateral flow test LFT can be used to detect an infection of a subject with legionellae.

It is not shown in FIG. 7 that the at least one or more proteins M can comprise MIP and/or PAL and/or FLA.

It is also not shown in FIG. 7 that the test system 10 is suitable for performing, at least in part, a method according to FIG. 17.

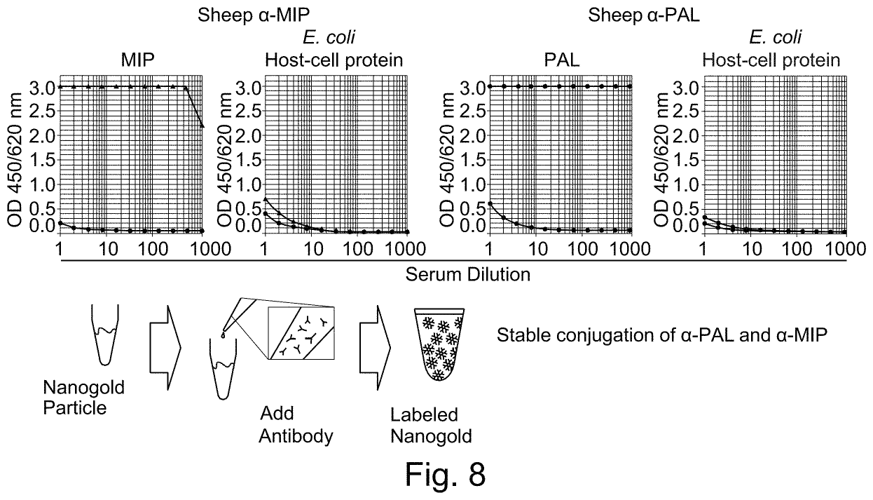

FIG. 8 shows the production of conjugation of gold N and antibodies B.

In particular, the production of stable immunoconjugates from antibodies B (sheep anti-PAL or sheep anti-MIP) and nanogold N is shown.

The production of stable immunoconjugates from anti-PAL can be carried out analogously.

FIG. 9 shows the stability of antigens in saliva.

The stability of bacterial antigens was studied by means of Western blot.

In particular, the stability of MIP and PAL in saliva was studied over a period of 10 min to 18 h (at room temperature, RT, or 37° C.).

The Western blot represents a method for the detection of proteins, e.g. in a protein mixture. In a first step, the protein mixture is separated into individual protein bands according to protein size, charge or other properties by gel electrophoresis in a support matrix (SDS-PAGE, native-PAGE, isoelectric focusing, 2D gel electrophoresis, etc.). The separated protein bands are then transferred from the gel to a solid support membrane (e.g. from nitrocellulose, nylon, or PVDF) for Western blotting. This process is called blotting. Due to charge interactions, proteins adhere to the membrane surface in the pattern of electrophoretic separation and are accessible to antibody binding for detection. The Western blot was carried out under standard conditions.

The antigens M MIP and PAL (recombinant proteins) were incubated in saliva (150 ng/μl).

The stability of MIP and PAL was studied with and without the addition of the non-specific protease inhibitor PMSF.

After the incubation, 600 ng of protein per line was applied.

Saliva (without the addition of recombinant protein, negative control) and recombinant protein (ng, positive control) were applied as controls.

A marker (molecular weight marker) was used to uniquely identify the marker proteins MIP and PAL.

The antigens M are stable in saliva both with and without protease inhibitor, in particular also at 37° C.

It is not shown in FIG. 9 that the stability of FLA in saliva can be measured analogously.

FIG. 10 shows the specificity of polyclonal α-MIP and α-PAL.

The specificity of polyclonal α-MIP (anti-MIP) and α-PAL (anti-PAL) was investigated by Western blot (compare FIG. 9).

In particular, the ability of α-MIP and α-PAL to detect different representatives of the oral microflora (Haemophilus parainfluenzae, Staphylococcus epidermidis, Rothia mucilaginosa, Neisseria perflava, Streptococcus mitis, Streptococcus salvarius, Legionella pneumophila) was investigated.

The pathogens were used as samples in the Western blot analysis.

A marker (molecular weight marker) was used to be able to uniquely identify the marker proteins M MIP and PAL by their size.

The recombinant proteins (antigens) MIP and/or PAL were used as positive controls.

Recombinant MIP was used as a further control for α-PAL and recombinant PAL for α-MIP.

Both antibodies showed no or only minimal, negligible cross-reactivity.

It is not shown in FIG. 10 that the analysis of the specificity of polyclonal α-FLA can take place analogously.

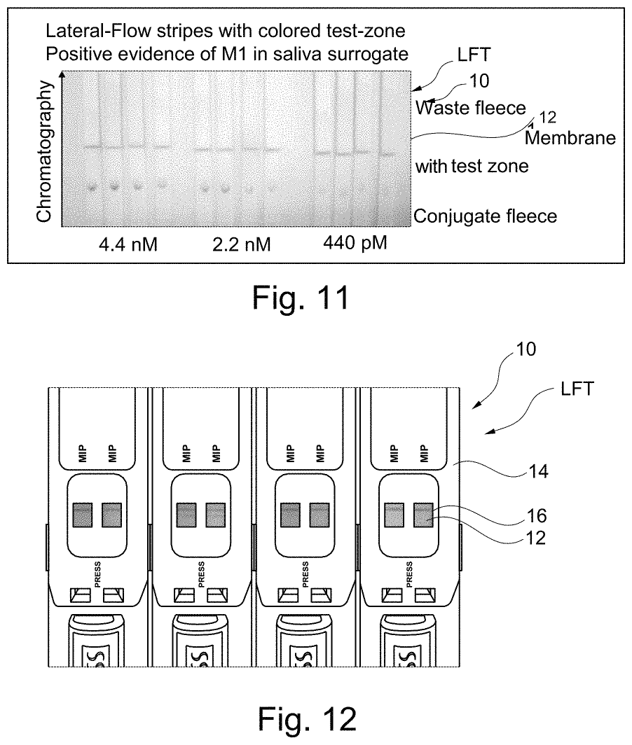

FIG. 11 shows an exemplary laboratory pattern of a lateral flow test LFT according to the disclosure.

The determination of the lower detection limit of simple lateral flow test strips LFT for MIP is shown (fourfold determination).

The principle of the test is essentially based on the system described in FIG. 7 (without control system 16 and sampling element).

A reddish spot on the membrane indicates specific detection of the marker protein M MIP.

Salivary surrogate was used as a sample.

In this case, the immunoconjugates are indicated as dots, not strips. The most sensitive test strips 12 achieved a sensitivity of approximately 2 ng/ml-20 ng/ml (440 pM) per marker protein M in saliva surrogate.

In a comparable test, the detection limit of PAL was determined. This limit is less than 2 ng/ml in saliva surrogate.

FIG. 12 shows test systems 10 for MIP each comprising a control system 16.

The test systems 10 function substantially according to the description in FIG. 7.

Four MIP development patterns are shown, each at the end of testing (approximately 20 minutes).

The test systems 10 each comprise an internal control system 16, which forms an additional band/line when the test (regardless of the test result) has been correctly performed and correctly run.

From left to right, test systems 10 are shown for the following samples: human saliva as negative sample, 5×10⁶ Legionella pneumophila. in saliva, 5×10⁵ Legionella pneumophila. in saliva, 5×10⁴ Legionella pneumophila in saliva.

The transfer of the sample from the sampling element to the test element 12 as well as the chromatography worked properly.

The control system 16 provided a signal after 10 minutes (upper of the two lines).

The lower detection limit of MIP is approximately 1×10⁵ intact Legionella pneumophila bacteria in human saliva per test element 12.

A (red) control line is visible in all tests, and an additional (red) test line is visible in the positive samples (greater than/equal to the detection limit).

The pattern does not show a false positive signal in the negative sample (even after extended readout time of up to two hours, not shown).

It is not shown in FIG. 12 that comparable results were obtained in analogous tests for the marker protein M PAL or FLA.

The lower detection limit for PAL is approximately 1×10⁷ intact Legionella pneumophila bacteria in human saliva per test strip.

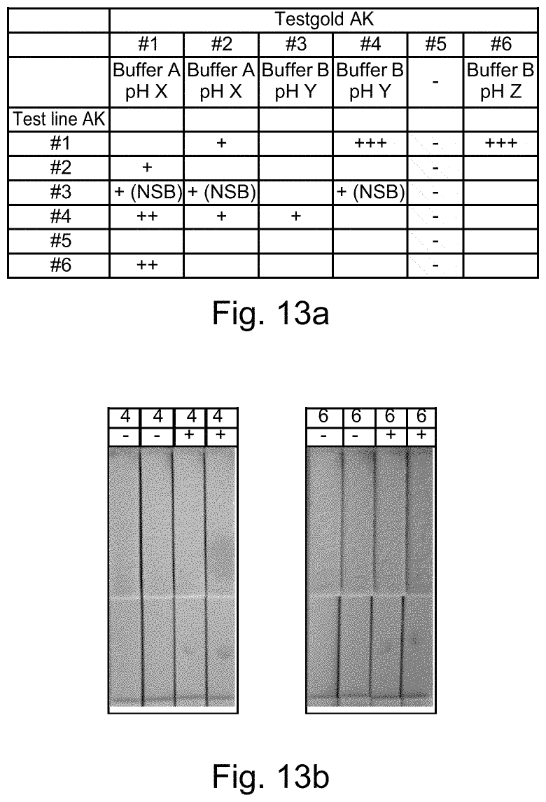

FIG. 13a and FIG. 13b show by way of example the selection of antibody pairings for PAL for use in a test system 10 according to the disclosure.

The aim is to select from the antibodies or immunoconjugates the pairs with which a sandwich assay can be built up for the lateral flow test (so-called “matching pairs”) (sandwich assay, cf. FIG. 7).

All available combinations (for PAL) of a test line antibody AK and an antibody nanogold conjugate (test gold AK) were built up on the membrane ( ) and the binding to the antigens (PAL) was investigated.

Dispensed test golds were used and test line AKs were spotted.

10 ng/ml PAL in pooled saliva was used as samples.

For the analyte PAL, it indicates that there are (at least) two matching antibody pairings:

Test gold AK #4 with test line AK #1 and vice versa test gold AK #1 with test line AK #4.

Test gold AK #6 with test line AK #1 and vice versa test gold AK #1 with test line AK #6.

+++ stands for strong binding, ++ for moderate binding, + for weak binding, NSB stands for non-specific binding (=false positive, means AK could not be tested (cf. FIG. 13a)).

FIG. 13b shows an example of a corresponding membrane for test line AK #1 with test gold AK #4 and #6; all tests in duplicate, “−” is the negative control (10 μl water), “+” is the positive sample with PAL (10 μl with c=100 ng/ml).

Test gold AK #1 is aggregated with nanogold. Therefore, it is necessary to find a stable test gold with this antibody to keep all combinations open.

It is not shown in FIG. 13a or 13b that using test gold antibodies (immunoconjugates) with gold nanoparticles from other manufacturers, it was possible to find further suitable antibody pairings.

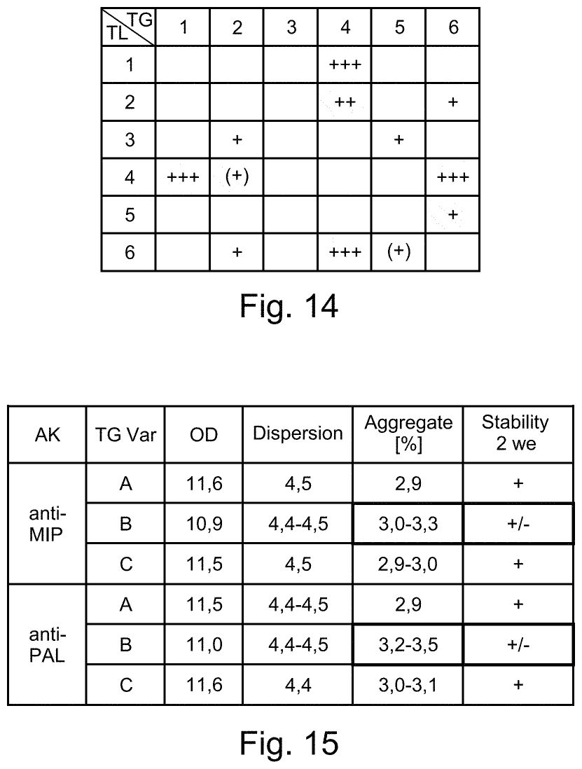

FIG. 14 shows an example of the selection of antibody pairings for MIP for use in a test system 10 according to the disclosure.

The selection procedure for MIP is identical in outline to the selection procedure for PAL, cf. FIGS. 13a and 13b.

Accordingly, the aim is to select from antibodies or conjugates the pairs with which a sandwich assay can be built up (so-called “matching pairs”). All available MIP combinations were built up in the form of spot tests. This was done on different membranes in conjunction with different RP systems.

The most suitable MIP pairings for the sandwich assay are:

Test gold (TG) AK #1 with test line AK #4 and vice versa test gold AK #4 with test line AK #1.

Test gold AK #6 with test line AK #4 and vice versa test gold AK+#4 with test line AK #6.

Furthermore: test gold AK #4 with test line AK #2 and vice versa test gold AK #2* with test line AK #4.

The pairing AK #5/AK #6 can also be used if, contrary to expectations, a sensitive and stable test cannot be built up with the above pairings.

FIG. 15 shows exemplary conditions for the conjugation of polyclonal antibodies and nanogold.

Several conditions were found from polyclonal anti-MIP or anti-PAL antibody and nanogold, under which several stable immunoconjugates can be prepared (buffer, pH, nanogold manufacturer, loading).

It is not shown in FIG. 15 that a competitive as well as a sandwich test basically work with the polyclonal AK and/or corresponding immunoconjugates.

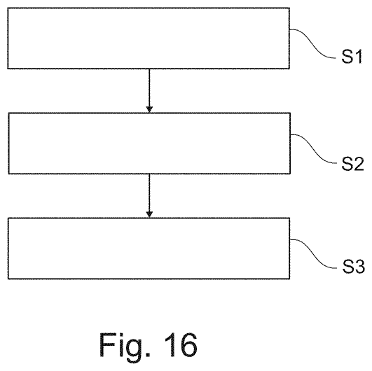

FIG. 16 schematically shows a method according to the disclosure for the detection of legionellae in a sample.

The method can be carried out at least in part with a test system 10 according to the disclosure, for example as shown in FIG. 7.

The method comprises at least steps S1-S3.

In a first step, S1, a test system 10 is provided.

The test system 10 comprises a test element 12.

In a second step, S2, a sample is applied to the test element 12.

In a third step, S3, the presence of at least protein M, in particular MIP, PAL and/or FLA, is detected in the sample.

The test system 10 comprises at least one specific binding partner B, to which the at least one protein M binds.

The detection is performed at least in part via the binding of the at least one protein M, in particular MIP, PAL and/or FLA, to the specific binding partner B.

Furthermore, the method can comprise the step of determining the amount of the at least one protein.

It is also possible that the method further comprises the step of lysing the sample.

Lysing can take place, for example, by means of detergents (e.g., Triton X-100), SDS and/or Tween) or using ultrasound.

It is additionally possible that the method further comprises the step of liquefying the sample.

The liquefaction can be performed at least in part by detergents and/or liquilizers.

The aim of liquefaction can be to allow the sample to better chromatograph via a lateral flow assay.

For example, the sample can be saliva or sputum.

The lower detection limit of MIP is approximately 1×10⁵ intact Legionella pneumophila bacteria in human saliva per test element 12.

The lower detection limit for PAL is about 1×10⁷ intact Legionella pneumophila bacteria in human saliva per test element 12.

### REFERENCE SIGNS

10 test system
12 test element/test strip
14 housing
16 control system
AK antibody
B specific binding partner for protein M
M protein/antigen
N labeling element
LFT lateral flow test/lateral flow assay
TG test gold/immunoconjugate antibody and nanogold
TL test lines antibody
S1 step 1
S2 step 2
S3 step 3

1. A test system for the identifying legionnaires' disease.

2. A test system for detecting infection of a subject with legionellae.

3. The test system according to claim 1, wherein the test system is or comprises a lateral flow test (LFT).

4. The test system according to claim 1, wherein the test system is set up to identify legionnaires' disease and/or to detect an infection with legionellae in saliva or sputum.

5. The test system according to claim 4, wherein the identification and/or detection takes place at least partially via the binding of at least one protein to a specific binding partner.

6. The test system according to claim 5, wherein the at least one protein is a protein of the species Legionella pneumophila, Legionella micadei, Legionella bozmanii, Legionella dumoffii and/or Legionella longbeachae.

7. The test system according to claim 6, wherein the at least one or also several proteins comprises MIP and/or PAL and/or FLA.

8. The test system according to claim 5, wherein the test system comprises:
a) a test element with microfluidic structures comprising:
(i) a first region set up to absorb eluent,
(ii) a second region set up for the application of a sample,
(iii) a third region set up for detecting the protein
(iv) a fourth region set up for absorbing excess eluent, and
b) a housing.

9. The test system according to claim 8, wherein the test element comprises a further region, the further region at least temporarily comprising the specific binding partner for the protein.

10. The test system according to claim 1, wherein the test system further comprises a control system.

11. The test system according to claim 5, wherein the test system further comprises a sampling element set up for receiving a sample containing the at least one protein.

12. A use of a test system according to claim 1 for detecting an infection with legionellae.

13. A method for detecting legionellae in a sample, comprising the following steps:
(a) providing a test system,
(b) applying the sample to a test element, and
(c) detecting the presence of at least one protein in the sample, wherein
the test system comprises at least one specific binding partner, to which the at least one protein binds,
wherein the detection is at least partly effected via the binding of the at least one protein to the specific binding partner.

14. The method according to claim 13, wherein the sample comprises a saliva sample or sputum.

15. The method according to claim 13, wherein the specific binding partner comprises a monoclonal antibody, polyclonal antibody, affinity-purified antibody, antibody fragment, peptide, protein, nanobody, nucleic acid fragment, aptamer, anticalin, lectin, affibody and/or chemical ligand, the specific binding partner being linked to a labeling element.

16. The method according to claim 15, wherein the labeling element comprises at least one dye, fluorochrome, fluorophore, luminophore, enzyme, peptide, biotin, oligonucleotide, protein tag, radioactive isotope, non-radioactive nuclide, nanoparticle.

17. The method according to claim 13, wherein the specific binding partner is available in soluble form and/or dried.

18. The test system according to claim 5, wherein the at least one binding protein is a protein of the bacterial genus Legionella.

19. The test system according to claim 8, wherein the test element a test element with microfluidic structures is a chromatographic test strip and wherein the third region is set up for optically detecting the proteins of MIP, PAL and/or FLA.

20. The test system according to claim 10, wherein the control system is an optical control system.

21. The labeling element according to claim 15, wherein the nanoparticle is gold nanoparticle, fluorescent nanoparticle, magnetic nanoparticle and/or quantum dot.
